(19) United States
(12) Patent Application Publication
(10) Pub. No.: US 2009/0131866 A1
(43) Pub. Date: May 21, 2009

(54) Veinous Occlusion Device and Methods of Using

(75) Inventors: Jianyi Zhang, Plymouth, MN (US); Carmelo Panetta, St. Paul, MN (US)

Correspondence Address: FISH & RICHARDSON P.C., PO BOX 1022, MINNEAPOLIS, MN, 55440-1022, US

(73) Assignee: REGENTS OF THE UNIVERSITY OF MINNESOTA, Minneapolis, MN (US)

(21) Appl. No.: 11/719,195

(22) PCT Filed: Nov. 10, 2005
(86) PCT No.: PCT/US2005/040723

§ 371 Date: Feb. 3, 2009

Related U.S. Application Data
(60) Provisional application No. 60/627,236, filed on Nov. 12, 2004.

(51) Int. Cl. A61M 29/00

(52) U.S. Cl. 604/96.01; 606/194

(57) ABSTRACT

The invention provides for methods of delivering compositions to an individual via the vasculature, and provides for a device that can be used to deliver compositions to an individual via the vasculature.

### TECHNICAL FIELD

This invention relates to a device for delivery of a composition to the vasculature system of an individual, and methods of using such a device to deliver a composition to the vasculature system.

### BACKGROUND

Investigations of cell transplantation for the injured heart typically deliver cells into or adjacent to a poorly perfused segment using intramyocardial injection. Such segments are largely necrotic, hypoxic, and infiltrated by macrophages and other immuno-responsive cells. Under these conditions, virtually all transplanted cells die and only less than 0.02% survive. Alternative delivery via coronary artery injection in a canine model caused significant microinfarctions in myocardial segments seeded by the cells. In contrast, the cardiac venous system has robust collateral vessels that afford resistance to such myocardial injury.

### SUMMARY

The invention provides for methods of delivering compositions to an individual via the vasculature, and provides for a device that can be used to deliver compositions to an individual via the vasculature.

In one aspect, the invention provides a device for occluding blood flow in a vessel. Such a device generally includes a catheter body and an occlusion member. The catheter body typically has a proximal portion and a distal portion that define a longitudinal axis, and an inner lumen. The occlusion member includes a bore through which the catheter body is engaged, and has a distal end and a proximal end.

A device of the invention can further include an outer sheath that is slidably movable along the longitudinal axis of the catheter body. A device of the invention can further include a catheter body sheath that is slidably movable along the longitudinal axis of the catheter body. Generally, the catheter body sheath is located between the catheter body and the outer sheath. In an embodiment, the distal end of the catheter body sheath is engaged with the proximal end of the occlusion member. A catheter body sheath can further include a catheter body sheath flange that is engaged with the proximal end of the occlusion member.

The occlusion member can be cylindrically-shaped or conically-shaped; and can be silicone or foam. In certain embodiments, the occlusion member is biodegradable. An occlusion member can be impregnated with a bioactive substance such as one or more growth factors or chemotherapeutic agents. A device of the invention can be disposable or sterilizable (i.e., reusable). A device of the invention (i.e., the inner lumnen of the catheter body) can include a composition selected from the group consisting of stem cells, one or more growth factors, one or more chemotherapeutic agents, a nucleic acid encoding a growth factor, and an anti-inflammatory compound.

In another aspect, the invention provides a method of delivering a composition to a biological target in an individual including: inserting and advancing the distal portion of the device of claim 1 into a vessel in the vasculature of the individual such that the distal portion of the device is positioned at a delivery site; retracting the outer sheath such that the occlusion member substantially occludes blood flow through the vessel; delivering the composition to the delivery site via the inner lumen of the catheter body; and removing the device from the vessel. Generally, the delivery site is distal to the occlusion member.

The method can further include discharging the occlusion member from the distal portion of the catheter body. Typically, the discharging step is prior to the removing step. In addition, it is desirable for optimal occlusion that the bore of the occlusion member closes following discharging of the occlusion member from the catheter body.

In certain embodiments, occlusion member is foam, silicone, or biodegradable, and can be cylindrically-shaped or conically-shaped. It is a feature of the invention that the vessel can be a vein. Representative compositions that can be delivered to a biological target include stem cells, one or more growth factors, one or more chemotherapeutic agents, a nucleic acid encoding a growth factor, and one or more anti-inflammatory compounds. Such stem cells can further include a heterologous nucleic acid encoding VEGF. Representative biological targets include the heart, liver, pancreas, kidney, brain, uterus, ovaries, prostate, testicles, intestines, eyes, vocal chord, and solid cancer tumors.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is an image of one embodiment of a veinous occlusion device in a retracted position.

FIG. 2 is an image of one embodiment of a veinous occlusion device in a deployed position.

FIG. 3A is a cylindrically-shaped occlusion member.

FIG. 3B is a conically-shaped occlusion member.

Like reference symbols in the various drawings indicate like elements.

### DETAILED DESCRIPTION

The invention provides for methods of delivering compositions to an individual via the vasculature, and provides for a device that can be used to deliver compositions to an individual via the vasculature.

### Veinous Occlusion Device

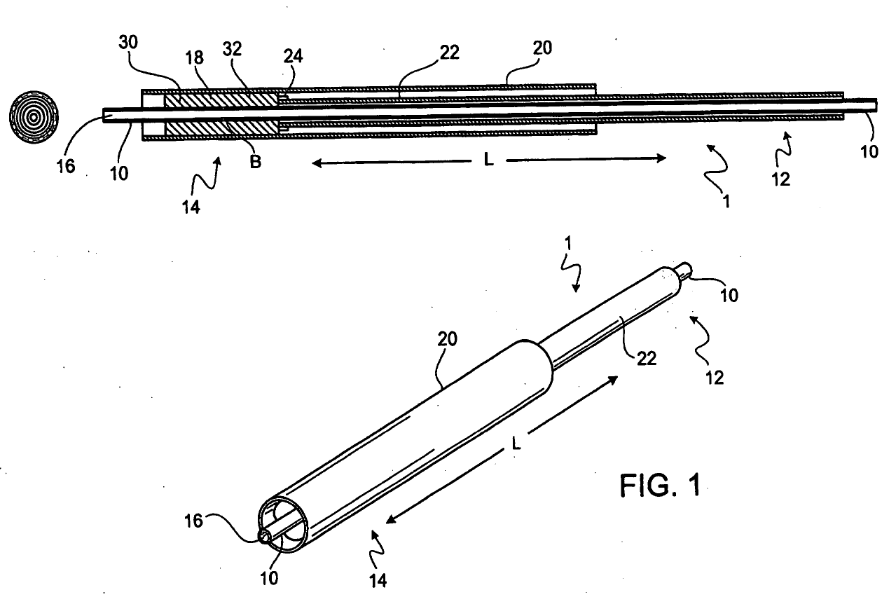

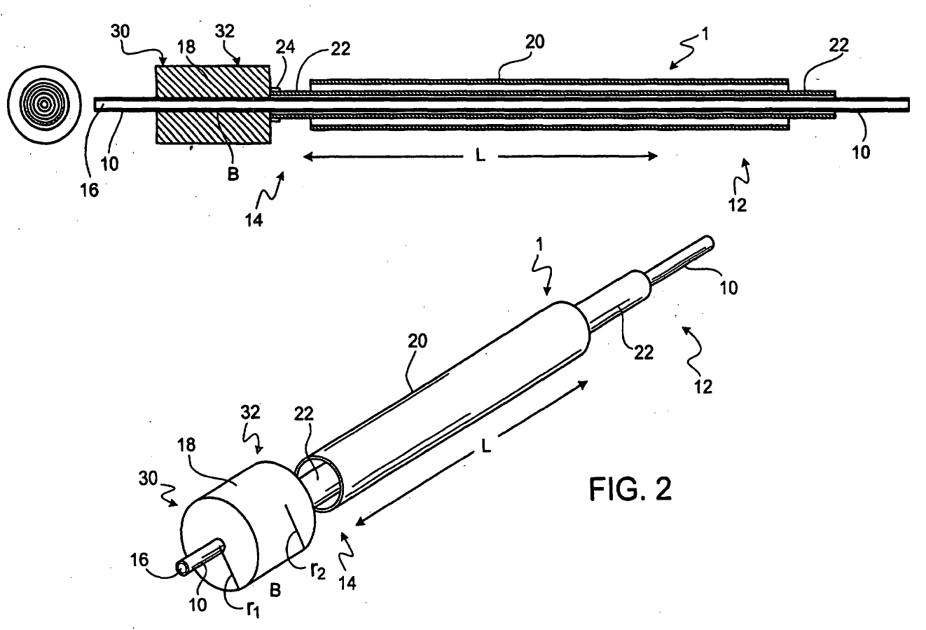

One embodiment of a veinous occlusion device 1 is shown in FIG. 1. The device shown in FIG. 1 is in the retracted configuration. A veinous occlusion device 1 includes a catheter body 10 having a proximal portion 12 and a distal portion 14. The proximal portion 12 and the distal portion 14 define a longitudinal axis L of the catheter body. A catheter body 10 suitable for use in a veinous occlusion device has an inner lumen 16. A veinous occlusion device 1 also can include an outer sheath 20 that is slidably moveable along the longitudinal axis L of the catheter body 10. FIG. 2 shows the device of FIG. 1 in the deployed configuration. According to the embodiment shown in FIGS. 1 and 2, deployment occurs by retracting the outer sheath 20 toward the proximal portion 12 of the catheter body 10, which exposes an occlusion member 18 that is engaged with the distal portion 14 of the catheter body 10. The occlusion member 18 shown in FIGS. 1 and 2 then expands to occlude blood flow.

The proximal 12 and distal 14 portions of the catheter body 10 can be integrally formed from a biocompatible material having requisite strength and flexibility for introducing and advancing a veinous occlusion device 1 of the invention into the vasculature of an individual. The proximal 12 and distal 14 portions can be flexible to facilitate articulation of the device during use. Appropriate materials are well known in the art and generally include polyamides such as, for example, a woven material available from DuPont under the trade name Dacron.

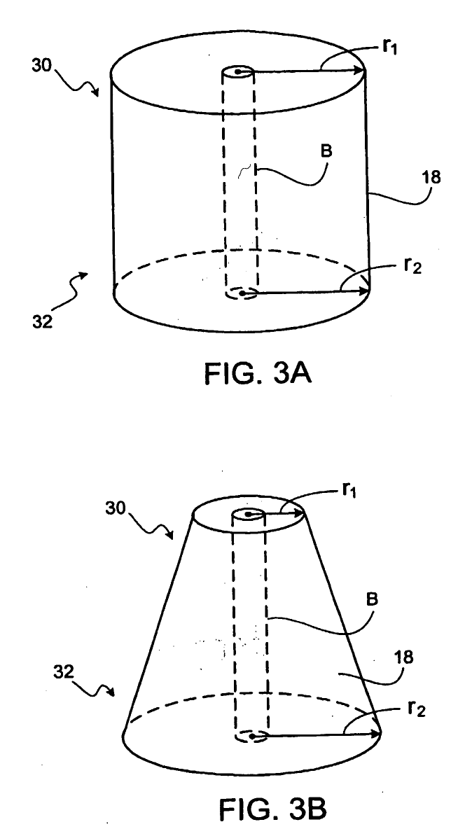

FIG. 3 shows different embodiments of an occlusion member. An occlusion member suitable for use in a veinous occlusion device 1 of the invention can have a bore B through which the distal portion 14 of the catheter body 10 extends. Generally, the size of the bore B correlates to the size of the distal portion 14 of the catheter body 10. An occlusion member 18 has a distal end 30 and a proximal end 32. In certain embodiments, the distal end 30 and/or the proximal end 32 can be substantially normal to the bore B. In another embodiment, the occlusion member can be solid (i.e., lack a bore), and the distal end of a catheter can be used to push out the occlusion member such that complete occlusion of the vessel occurs.

The radius of the distal end (r₁) and the radius of the proximal end (r₂) can vary relative to one another. For example, the radius of the distal end (r₁) and of the proximal end (r₂) can be substantially equal resulting in a cylindrically-shaped occlusion member (r₁≅r₂) (FIG. 3A), or the radius of the distal end (r₁) and of the proximal end (r₂) can be different resulting in a conically-shaped occlusion member (r₁>r₂ or r₁<r₂) (FIG. 3B). The maximal circumference of a deployed occlusion member should be compatible with the size of vessel into which the occlusion member is being introduced. As used herein, “compatible with” refers to an occlusion member that, when deployed, is seated tightly against the vessel wall for optimal occlusion but is not large enough to disrupt or compromise the vessel.

An occlusion member can be made from any number of materials. In certain embodiments, the occlusion member is initially compressed, and then expands following retraction of the outer sheath. Representative materials that are compressable and/or expandable include, without limitation, foam and silicone. Any other material that allows for deployment and subsequent occlusion of a vessel is suitable for use in an occlusion member provided that the material can tolerate having a bore therethrough. In addition, occlusion members can be made from biodegradable materials.

Many biodegradable materials are derived from renewable resources such as starch, cellulose, and polyhydroxyalkanoates, and from synthetic means such as polylactic acid and polycaprolactone. Polyhydroxyalkonates are a family of naturally occurring polyesters that are produced in the form of carbon storage granules by many bacteria. One commercially available product is BIOPOL™. In addition, products based on lactic and glycolic acid as well as other materials including poly(dioxanone), poly(trimethylene carbonate) copolymers, and poly (ε-caprolactone) homopolymers and copolymers, polyanhydrides, polyorthoesters, and polyphosphazenes are either currently used in medical devices or are being developed for use in medical devices. Another biodegradable material that can be used in the methods or the device of the invention is the fibrin biomatrix disclosed in U.S. application Ser. No. 10/874,449.

An occlusion member can be impregnated with one or more bioactive substances. For example, an occlusion member can be impregnated with a growth factor (discussed below) or with chemotherapeutic agents (discussed below). Occlusion members also can be impregnated with any other bioactive drug or molecule having beneficial clinical effects on the biological target. Such bioactive substances can be engineered for slow release over time from the occlusion member, or if present in a biodegradable occlusion member, the bioactive substance is released at approximately the same rate as the biodegradation of the occlusion member.

After deployment of the occlusion member, the occlusion member can be discharged from the catheter body and can remain in the vessel occluding blood flow for hours, days, or weeks. FIGS. 1 and 2 show one embodiment of a discharge mechanism, although any means to discharge the occlusion member can be used. For example, FIGS. 1 and 2 show a veinous occlusion device 1 that includes a catheter body sheath 22 and a catheter body sheath flange 24. A catheter body sheath 22 and a catheter body sheath flange 24 can be used to discharge the occlusion member 18. To achieve complete or almost complete occlusion, it is desirable that the bore B close itself or collapse in on itself after the catheter body has been removed. A catheter body sheath 22 and a catheter body sheath flange 24 also can be used to maintain the position of the occlusion member 18 on the catheter body 10 during retraction of the outer sheath 20.

A veinous occlusion device 1 of the invention can optionally include a device for imaging or monitoring at the delivery site. For example, an intracardiac echo (ICE) device can be used to image a vessel (e.g., for appropriate positioning of the occlusion device) or to measure the diameter of a vessel. Other imaging or monitoring devices or elements can be used such as an ultrasound assembly or sensing elements such as electrodes. A device for imaging and/or monitoring can be attached to a veinous occlusion device 1 at the distal portion 14 of the catheter body 10.

### Compositions for Delivery by Veinous Occlusion Device

Cells having an established function can be delivered to a particular biological target to facilitate repairs or improve the function of a particular tissue (e.g., heart, lung, skin, bone, liver, kidney, pancreas, testis, and ovary). For example, pancreatic beta cells can be delivered to the pancreas to improve pancreatic function in an individual. Other cell types include, without limitation, islet cells, epithelial cells, endothelial cells, hepatocytes, nephrocytes, glomerulocytes, osteocytes (e.g., osteoblasts and osteoclasts), lymphocytes (e.g., T cells, B cells, and NK cells), granulocytes (e.g., neutrophils, basophils, eosinophils, and mast cells), and fibroblasts. In addition, cell types that have been engineered to perform a particular function, such as genetically altered cells, can be delivered to a biological target.

Cells having the ability to differentiate into various cell types also can be delivered to a biological target. Such cells include, without limitation, stem cells and progenitor cells. Stem cells are cells with extensive proliferation potential that can differentiate into several cell lineages. For example, embryonal stem (ES) cells have unlimited self-renewal and multipotent differentiation potential. ES cells are derived from the inner cell mass of the blastocyst, or can be derived from primordial germ cells from a post-implantation embryo (embryonal germ cells or EG cells). Stem cells have been identified in many tissues. Typical stem cells include, without limitation, hematopoietic, neural, gastrointestinal, epidermal, hepatic, mesenchymal stem cells (MSCs), stem cells from exfoliated deciduous teeth, and autologous bone marrow stem cells (ABMSCs). Progenitor cells have multipotent differentiation and extensive proliferation potential. Progenitor cells can differentiate in vitro into most mesodermal cell types including cells with characteristics of skeletal and cardiac myoblasts, as well as cells with endothelial and smooth muscle features. Any combination of stem cells, progenitor cells, or other types of cells can be delivered to a biological target.

Stem or progenitor cells can be obtained from various species including, without limitation, mouse, rat, dog, pig, cow, goat, horse, non-human primates, and humans. Although allogeneic and xenogeneic cells are within the scope of the invention, autologous stem or progenitor cells are typically used. Stem or progenitor cells can be isolated from various tissues of an individual including, without limitation, brain, spinal cord, lung, skin, liver, blood, and bone marrow. For example, stem cells can be isolated from bone marrow aspirated from an individual. Briefly, a needle is used to penetrate the outer core of a bone (e.g., the iliac crest) in an anesthetized individual. When using a syringe, negative pressure is applied by forcefully withdrawing the syringe plunger, allowing the marrow to be collected in the syringe barrel. The marrow is then layered onto a gradient substrate (e.g., Ficoll) in a conical tube. The marrow is then centrifuged to collect autologous bone marrow mononuclear cells at a known interface. After subsequent culture selection, the ABMSCs can be manipulated (e.g., transfected with a plasmid or transduced with a virus) prior to use. Alternatively, techniques such as those disclosed in U.S. Pat. Nos. 5,486,359 and 6,261,549 also can be used for isolating, purifying, and characterizing stem and progenitor cells suitable for use in the invention.

Stem cells and progenitor cells can be engineered ex vivo to augment their therapeutic value. For example, vascular endothelial growth factor (VEGF) is a multi-functional growth factor that regulates cell proliferation, migration and survival. Within sites of neo-angiogenesis, VEGF has been shown to promote the mobilization and recruitment of various progenitor cells that accelerate the revascularization process.

Chemotherapeutic agents also can be delivered to a biological target using a veinous occlusion device of the invention. Without limitation, chemotherapeutic agents include antineoplastic and cytotoxic agents, immunosuppressants, antiviral medications, and any other compounds that can be used to treat cancer. Most chemotherapeutic agents or combinations thereof have the ability to kill cancer cells. Examples include busulfan, cisplatin, cyclophosphamide, methotrexate, daunorubicin, doxorubicin, melphalan, cladribine, vincristine, vinblastine, and chlorambucil. Chemotherapeutic agents generally are administered to an individual in a particular regimen over a period of weeks or months.

In addition to the VEGF discussed above, other growth factors also can be delivered to a biological target using a veinous occlusion device of the invention. Growth factors generally are proteins that bind to receptors on the surface of a cell and activate cellular proliferation and/or differentiation. Representative growth factors include, for example, epidermal growth factor (EGF), platelet-derived growth factor (PDGF), fibroblast growth factor (FGF), transforming growth factors -α and -β (TGF-α and TGF-β), erythropoietin (Epo), and insulin-like growth factor-I and -II (IGF-I and IGF-II). Alternatively, one or more nucleic acids encoding one or more growth factors can be delivered to a biological target using a veinous occlusion device of the invention.

Anti-inflammatory compounds also can be delivered to a biological target using the methods and/or device described herein. Anti-inflammatory compounds are compounds that prevent or reduce inflammation. The most common anti-inflammatory compounds include non-steroidal anti-inflammatory drugs (NSAIDs), although numerous other anti-inflammatory compounds and proteins (e.g., viral encoded proteins) are known in the art.

According to the invention, a delivery site is within the vasculature, while a biological target is any organ or tissue through which vasculature passes. For example, biological targets can include, without limitation, the heart, liver, pancreas, kidney, brain, uterus, ovaries, prostate, testicles, intestines, eyes, and vocal chord. In addition, a biological target can include a solid tumor or mass virtually anywhere in an individual.

### Methods of Using A Veinous Occlusion Device

A veinous occlusion device of the invention can be used to deliver a composition to a target biological. Typically, the distal end of the catheter body is inserted and advanced into the vasculature of an individual and positioned relative to a delivery site such that upon deployment, the occlusion member is occluding blood flow on the distal side of the occlusion member (relative to the operator). Any of the compositions described above can be delivered to a delivery site and ultimately to a biological target via the inner lumen of the catheter body.

Inserting and advancing a catheter into the vasculature on an individual are well-known and routine techniques used in the art. The “Seldinger” technique is routinely used for introducing a sheath such that a catheter can be advanced into the right venous system of an individual. It is contemplated, however, that other methods for introducing a veinous occlusion device of the invention into a vessel are suitable and include, for example, a retrograde approach or a venous cut-down approach.

The veinous occlusion device shown in FIG. 1 is in the retracted configuration. It is in this retracted configuration that the device would be introduced into an individual. Once the distal portion of the catheter body is positioned an appropriate distance on the proximal side of the delivery site (relative to the device operator), the occlusion member can be deployed. Upon deployment of the occlusion member, blood flow is substantially occluded. “Substantially occluded” refers to decreasing blood flow by at least 30% (e.g., by 35%, 40%, 50%, 60%, 70%, 80%, 90%, 95%, 99%, or 100%).

There are numerous advantages to delivering compositions via the veinous vasculature. One advantage of the present invention is that since the blood flow typically is blocked on the side of the occlusion member containing the delivery site (distal to the occlusion member relative to the device operator), there is essentially no blood flow to wash away the delivered composition. As a veinous occlusion can be tolerated for hours, days, weeks, months, or even longer, the composition has time to assimilate into a biological target without being washed away or disrupted by blood flow. This aspect of the invention cannot be appreciated with arterial delivery, in which blood flow can be stopped for only minutes or seconds.

### Articles of Manufacture

A veinous occlusion device of the invention can be packaged in a number of ways. For example, a veinous occlusion device of the invention can be manufactured and packaged for a single use (i.e., disposable). Alternatively, a veinous occlusion device of the invention can be manufactured to be reusable and sterilizable. In embodiments in which the device is sterilizable, additional occlusion members can be provided in conjunction with a device, or they can be provided separately. In addition, a variety of different occlusion members (e.g., different materials, different sizes, and/or different shapes) can be packaged and provided to a user.

The invention can include an article of manufacture (e.g., a kit) that contains a composition for delivery to a biological target (e.g., one or more chemotherapeutic agents). Articles of manufacture also can contain a package insert or label having instructions thereon for using such a composition. An article of manufacture of the invention also can contain the materials necessary for obtaining stem cells or progenitor cells from an individual, and may additionally include a package insert or label having instructions thereon for collecting stem or progenitor cells from an individual. Methods and materials for obtaining and preparing stem cells or progenitor cells have been discussed herein.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### Materials and Methods

All procedures and protocols were approved by the University of Minnesota Animal Care Committee. The investigation conformed to the Guide for the Care and Use of Laboratory Animals by the Institute of Laboratory Animal Research (Institute of Laboratory Animal Resources, 1996, Guide for the care and use of laboratory animals, 7th ed. Washington, D.C., National Academy Press). In this study, 7 pigs received VEGF-modified MSCs (VEGF−MSCs) via cardiac vein injection for comparison against 19 untreated LVH and 8 normal pigs. Replication-deficient recombinant adenoviruses carrying the nuclear β-galactosidase reporter gene lacZ was purchased from the University of Iowa Gene Vector Core). Swine VEGF165 expression vector was kindly provided by Dr. John Canty (University of Buffalo).

### Example 2

### Swine Mesenchymal Stem Cell Culture

MSCs from bone marrow were isolated by gradient density centrifugation (Liu et al., 2004, Am. J. Physiol. Heart Circ. Physiol., 287:H501-11; Pittenger et al., 1999, Science, 284:143-7). Bone marrow was aspirated from the sternum of healthy Yorkshire pigs into a syringe containing 6000 U heparin, and diluted with Dulbecco's PBS in a ratio of one to one. The marrow sample was carefully layered onto the Ficoll-Paque-1077 (Sigma) in a 50 ml conical tube and centrifuged at 400×g for 30 min at room temperature. The mononuclear cells were collected from the interface, washed with 2-3 volumes of Dulbecco's PBS and collected by centrifugation at 1000 rpm. The cells were resuspended and seeded at a density of 200,000 cells/cm² in T-75 flask coated with 10 ng/ml fibronectin (FN) and cultured in medium consisting of 60% low-glucose DMEM (Gibco BRL), 40% MCDB-201 (Sigma), 1×insulin transferin selenium, 1×linoleic acid bovine serum albumin (LA-BSA), 0.05 μM dexamethasone (Sigma), 0.1 mM ascorbic acid 2-phosphate, 2% FCS, 10 ng/ml PDGF, 10 ng/ml EGF, 10 U/ml penicillin and 100 U/ml streptomycin. After 3 days, nonadherent cells were removed by replacing the medium. The attached cells grew and developed colonies in about 5-7 days. After approximately 10 days, the primary cultures of MSC reached nearly 90% of confluence; cells were subcultured by incubation with trypsin. The first passage cells were plated at 4000-5000 cells/cm² and further cultured two days for the transduction with VEGF or AdRsvLacZ.

### Example 3

### Cell Phenotype

CD44, CD45, CD90, MHC-Class I, MHC-Class II, SWC3A and SLA-DR were detected by flow cytometry. 0.5−1×10⁶ MSCs were placed in 100 μl BSA/PBS solution for each phenotype test and incubated with 2 μg primary mouse monoclonal antibodies (mAbs) against pig CD44, CD45, CD90, MHC-Class I, MHC-Class II, SWC3A and SLA-DR for 40 min at 4° C. The second polyclonal antibody IgG against mouse, FITC conjugated, (1 μg/tube) was added and incubated at 4° C. for an additional 30 min in a dark room. 2 μg of mouse IgG instead of primary mAbs was added to 0.5−1×10⁶ cells for a negative control.

### Example 4

### Adenoviral Transduction

VEGF was subcloned into the shuttle vector pacAd5CMVK-Np. Viruses were prepared and titrated by the gene vector Core Lab at University of Iowa. Adenovirus infections were performed 24 h after plating. Cells were incubated for 3 h at 37° C. with 0.5 ml of serum free culture medium containing the virus at the appropriate concentration, and then re-fed with fresh 2% serum medium. Viral concentrations used for transduction of the nuclear β-galactosidase reporter gene lacZ were described previously (Liu et al., supra).

### Example 5

### Induction of Left Ventricular Hypertrophy and Cell Transplantation

A swine model of severe concentric LVH/CHF was produced as previously described (Mangi et al., 2003, Nat Med., 9:1195-1201; Ye et al., 2001, Circulation, 103:1570-1576; Zhang et al., 1993, J. Clin. Invest., 92:993-1033). Briefly, Yorkshire pigs at ˜45 days of age were anesthetized with sodium pentobarbital (25-30 mg/kg iv), intubated and mechanically ventilated. A right thoracotomy was performed through the third intercostal space, and the ascending aorta was encircled with a polyethylene band 2.5 mm in width at approximately 1.5 cm above the aortic valve. While simultaneously measuring left ventricular and distal aortic pressures, the band was tightened until a 55-60 mmHg-peak systolic pressure gradient was achieved across the narrowing. A silicone elastomer catheter (1.0 mm i.d.) was inserted into the interventricular vein (great cardiac vein). The vein was proximally occluded, and ˜30 million VEGF−MSCs were slowly injected through the catheter. The catheter was then removed and the intracoronary vein close to the injection site was repaired. The chest was closed in layers, and the animal was allowed to recover. LVH developed progressively as the area of aortic constriction remained fixed in the face of normal body growth. 25 days after banding, the animals were returned to the laboratory for MRI and spectroscopic and hemodynamic measurements.

### Example 6

### Animal Preparation for MRI and Spectroscopic and Hemodynamic Measurements

Animal preparation for spectroscopic and hemodynamic measurements was described in detail previously (Ye et al., supra). All MRI studies were performed on a Siemens Medical VISION® System operating at 1.5 Tesla within 3 days of the final MRS and physiological study. The animals were sedated with ketamine (20 mg/kg i.m.), anesthetized with sodium pentobarbital (30 mg/kg, i.v.), intubated and ventilated with a respirator. Animals were placed on their left side in an 18 cm diameter Helmholtz coil. Imaging sequences were gated to the ECG while respiratory gating was achieved by triggering the ventilator to the cardiac cycle between data acquisitions. A detailed account of the imaging and analysis methodologies, including determination of left ventricular chamber volumes and ejection fractions, has been reported previously (Murakami et al., 1999, Circulation, 99:942-8; and Zhang et al., 1996, Circulation, 94:1089-1100). LV systolic wall thickening fraction (ST %) was measured at anterior wall using the equation: ST %=100%×(lₛ−l_{d})/l_{d}; where: lₛ=LV thickness_{systole}(mm), l_{d}=LV thickness_{diastole}(mm).

### Example 7

### Myocardial Blood Flow

Myocardial blood flow was measured using 15-μm diameter microspheres labeled with gamma-emitting radionuclides (¹⁴¹Ce, ⁵¹Cr, ⁹⁵Nb, ⁸⁵Sr or ⁴⁶Sc) as described previously (Domenech et al., 1969, Circ. Res., 25:581-596; Ye et al., supra; Zhang et al., 1996, Circulation, 94:1089-1100).

### Example 8

### Experimental Preparation for MRS Study

Animals were anesthetized with sodium pentobarbital (30 mg/kg iv.), intubated and ventilated with a respirator with supplemental oxygen. Arterial blood gases were maintained within the physiologic range by adjustments of the respirator settings and oxygen flow. A heparin-filled polyvinyl chloride catheter (3.0 mm OD) was introduced into the right femoral artery and advanced into the ascending aorta. A sternotomy was performed and the heart suspended in a pericardial cradle. A second heparin-filled catheter was introduced into the left ventricle through the apical dimple and secured with a purse string suture. A similar catheter was placed into the left atrium through the atrial appendage. A 25 mm diameter NMR surface coil was sutured onto the left ventricular anterior wall. The pericardial cradle was then released and the heart was returned to its normal position. The surface coil leads were connected to a balanced-tuned circuit external and perpendicular to the thoracotomy incision. The animals were then placed in a Lucite cradle and positioned within the magnet.

### Example 9

### Spatially Localized ³¹P NMR Spectroscopic Technique

Measurements were performed in a 40 cm bore 4.7 T magnet interfaced with a SISCO (Spectroscopy Imaging Systems Corporation, Fremont, Calif.) console. The left ventricular pressure signal was used to gate NMR data acquisition to the cardiac cycle while respiratory gating was achieved by triggering the ventilator to the cardiac cycle between data acquisitions (Liu & Zhang, 1999, J. Magn. Reson. Imaging, 10:892-8; and Zhang et al., 1993, J. Clin. Invest., 92:993-1003). ³¹P and ¹H NMR frequencies were 81 and 200.1 MHz, respectively. Spectra were recorded in late diastole with a pulse repetition time of 6-7 seconds. This repetition time allowed full relaxation for ATP and inorganic phosphate (Pi) resonances, and approximately 95% relaxation for the PCr resonance (Liu & Zhang, supra). Creatine phosphate (PCr) resonance intensities were corrected for this minor saturation; the correction factor was determined for each heart from two spectra recorded consecutively without transmural differentiation, one with 15 second repetition time to allow full relaxation and the other with the 6 second repetition time used in all the other measurements.

Radio frequency transmission and signal detection were performed with a 25 mm diameter surface coil. The coil was cemented to a sheet of silicone rubber 0.7 mm in thickness and approximately 20% larger in diameter then the coil itself. A capillary containing 15 μl of 3 M/L phosphonoacetic acid was placed at the coil center to serve as a reference. The proton signal from water detected with the surface coil was used to homogenize the magnetic field and to adjust the position of the animal in the magnet so that the coil was at or near the magnet and gradient isocenters. This was accomplished using a spin-echo experiment and a readout gradient. The information gathered in this step was also utilized to determine the spatial coordinates for spectroscopic localization (Liu & Zhang, supra). Chemical shifts were measured relative to PCr which was assigned a chemical shift of −2.55 ppm relative to 85% phosphoric acid at 0 ppm (Zhang et al., supra).

Spatial localization across the left ventricular wall was performed with the RAPP-ISIS/FSW method (Liu & Zhang, supra). Detailed data with regard to voxel profiles, voxel volume and extensive documentation of the accuracy of the spatial localization obtained in phantom studies and in vivo have been published elsewhere (Liu & Zhang, supra). Briefly, signal origin was restricted using B₀ gradients and adiabatic inversion pulses to a column coaxial with the surface coil perpendicular to the left ventricular wall. The column dimensions were 17 mm×17 mm. Within this column, the signal was further localized using the B₁ gradient to 5 voxels centered about 45°, 60°, 90°, 120°, and 135° spin rotation increments (Liu & Zhang, supra; and Zhang et al., supra). FSW localization utilized a 9-term Fourier series expansion. The Fourier coefficients, number of free induction decays acquired for each term in the Fourier expansion and the multiplication factors employed to construct the voxels have been reported previously (Liu & Zhang, supra). The position of the voxels relative to the coil was set using the B₁ magnitude at the coil center which was experimentally determined in each case by measuring the 90° pulse length for the phosphonoacetic acid reference located in the coil center. Each set of spatially localized transmural spectra were acquired in 10 minutes. A total of 96 scans were accumulated within each 10 minute block.

Resonance intensities were quantified using integration routines provided by the SISCO software. ATPγ resonance was used for ATP determination. Since data were acquired with the transmitter frequency positioned between the ATPγ and PCr resonance, off resonance effects on these peaks were virtually non-existent. The numerical values for PCr and ATP in each voxel were expressed as ratios of PCr/ATP. Inorganic phosphate (Pᵢ) levels were measured as changes from baseline values (ΔPᵢ), using integrals obtained in the region covering the Pᵢ resonance.

### Example 10

### Experimental Protocol

Hemodynamic measurements and ³¹P NMRS spectra were first obtained under basal conditions. Midway through the 10 minute NMRS acquisition period a microsphere injection was performed for determination of myocardial blood flow. Arterial blood gases were measured every 10 minutes, and the respirator was adjusted to maintain the normal physiologic pO₂, pCO₂ and pH. After baseline data were obtained, dobutamine and dopamine were infused simultaneously (each 20 μg/kg/min i.v.) to induce a high cardiac workstate (HCW). After allowing ˜10 minutes to achieve a steady state, all measurements were repeated.

### Example 11

### Cell Engraftment Rate Determination

Four weeks after cell transplantation, every heart was cross-sectioned into 8 to 10 rings. Odd number rings were used to determine the cell engraftment rate and histology analysis, and even number rings were snapping frozen for QRT-PCR. For histological analysis every ring was divided into 10 to 12 pieces. After X-gal staining, tissues were embedded in Tissue-Tek OCT compound (Fisher Scientific), and frozen in a liquid nitrogen-cooled isopentane. 10-μm thick frozen tissue sections were sectioned on a cryostat. Total cell nuclei were stained with DAPI (4′, 6-diamidino-2-phenylindole; Sigma-Aldrich). The engraftment cell number was analyzed by X-gal and DAPI double positive nuclei in every 10th serial sections.

### Example 12

### RNA Isolation and cDNA Preparation

Snap-frozen LV specimens were pulverized in liquid nitrogen. Total RNA was isolated using RNeasy columns with RNase-free DNase treatment. 1 μg total RNA was used for reverse transcription reactions using oligo (dT)₁₈ as a primer.

### Example 13

### Quantitative Real-Time RT-PCR

Changes in mRNA levels under different experimental conditions were compared by quantitative real-time RT-PCR analysis using the LightCycler™ thermocycler (Roche Diagnostics Corp) as previously described (Wang et al., 2002, Circ. Res., 90:340-347). Primer sequences and reaction parameters are depicted in Table 1.

TABLE 1

| | Ta* | Te* | Extension Time | | SEQ ID |
|---|---|---|---|---|---|
| Gene | (° C.) | (° C.) | (sec) | Primer Sequences | NO: |
| GAPDH | 60 | 72 | 18 | 5′-ACCACAGTCCATGCCATCAC-3′ | 1 |
| | | | | 5′-TCCACCACCCTGTTGCTGTA-3′ | 2 |
| VWF | 65 | 72 | 15 | 5′-GCTGCCACGCCTACATCTCG-3′ | 3 |
| | | | | 5′-TCCACACCGCTGACCACAAAG-3′ | 4 |
| VEGF | 62 | 72 | 16 | 5′-CCTTGCCTTGCTGCTCTACC-3′ | 5 |
| | | | | 5′-TTGCCTCGCTCTATCTTTCTTTG-3′ | 6 |

*Ta, annealing temperature; Te, extension temperature

### Example 14

### BrdU analysis

The function of VEGF secreted by the transduced MSCs was assessed via a BrdU incorporation assay (Boehringer Mannheim, Tokyo) of endothelial cells cultured in media conditioned by VEGF−MSCs.

### Example 15

### Immunohistochemistry and Immunofluorescenses

Tissue samples were cryoprotected in cold 2-methylbutane for 1 hour, embedded in Tissue-Tek OCT (Fisher Scientific), and sectioned into 10 μm slices using a cryostat. Immunohistochemistry and immunofluorescence staining were performed as previously described (Wang et al., supra). The following primary antibodies were used: mouse anti-human vWF and mouse anti-mouse caveolin-1 antibodies (BD Biosciences), Troponin T antibody (NeoMarkers), mouse anti-canine phospholamban and mouse anti-human alpha myosin heavy chain antibodies (Abcam), and fluorescence-labeled secondary antibodies (Molecular Probes).

### Example 16

### VEGF−MSCs Conditioned Medium and Myocytes Apoptosis

HL-1 myocytes (from Claycomb Laboratory, University of Louisiana) were plated onto fibronectin-gelatin-coated plates or flasks and cultured in Claycomb medium supplemented with 10% fetal bovine serum, 100 units/ml penicillin, 100 μg/ml streptomycin, 0.1 mM norepinephrine and 2 mM L-glutamine as previously described (Claycomb et al., 1998, PNAS USA, 95:2979-84; and White et al., 2004, Am. J. Physiol. Heart Circ. Physiol., 286:H823-9). For the conditional medium experiment, swine MSCs were transfected with 100 pfu/cell of VEGF adenovirus and nuclear LacZ adenovirus. Six hours after infection, cells were washed three times and placed in MSC culture medium as previously described (Liu et al., supra). The conditioned medium was harvested 48 hours after culture. As a control, a portion of the conditional medium was incubated for 48 hours without MSCs. HL-1 cells were cultured in the conditioned medium and exposed to 2% oxygen (hypoxia) for 24 hours to induce apoptosis.

For the co-culture experiment, HL-1 cells were plated in 12 well plates at a total density of 5×10⁵ (1:30 ratio; MSCs:HL-1 cells) in half MSC medium and half Claycomb medium. Prior to co-culture, cells were labeled with Vybrant CFDA SE cell tracer kit (Molecular Probes). Labeled HL-1 cells were extensively washed and co-cultured with MSCs or VEGF-modified MSCs. The cells then incubated for 24 hours at 2% oxygen (hypoxia) or 21% oxygen (normoxia). Apoptosis was assessed by staining with Hoechst 33342 (H33342) dye and then quantifying the percentage of apoptotic nuclei (300 cells counted/sample) in the CFDA labeled subset by identifying cells (Wang et al., 2004, Am. J. Physiol. Heart Circ. Physiol., 287:H2376-83; and Wang et al., 2002, Circ. Res., 90:340-7).

### Example 17

### Data Analysis

One-way ANOVA identified the presence of any differences-in-group means. The Scheffe multiple comparison test evaluated the significance of pairwise differences between group means. Differences were considered statistically significant at a value of p<0.05.

### Example 18

### Genetically Engineered Autologous VEGF-Overexpressing MSCs

MSCs isolated from adult swine bone marrow were positive for CD90, CD44, SWC3A and HLA-Class I; and negative for CD34, CD45 and HLA-Class II. Adenoviral transduction of MSCs with both swine VEGF165 DNA and the lacZ control was 90% efficient. Real-time quantitative RT-PCR detected both endogenous and exogenous porcine VEGF using specific probes designed. VEGF−MSCs transduced at 10 pfu/cell and 100 pfu/cell expressed VEGF mRNA at levels 10 and 30 times greater than the endogenous levels of lacZ-MSCs, respectively. These results indicated successful integration of the exogenous VEGF gene into the genome of the porcine MSCs. Moreover, immunohistochemistry showed significantly increased expression of VEGF in transduced MSCs.

To assess the function of VEGF secreted by VEGF−MSCs, a 5-bromo-2′-deoxyuridine (BrdU) incorporation assay was performed using human umbilical endothelial cells (HUVECs) cultured in media conditioned by VEGF−MSC. Briefly, HUVECs were cultured in normal MSC medium for 24 hours followed by 24 hours in serum-free medium. Separate HUVEC flasks received conditioned medium obtained from either VEGF or lacZ-only transduced MSCs, and were labeled for 6 or 12 hrs using 10 μl of BrdU solution (1 mM BrdU in Dulbecco's phosphate-buffered saline). A negative control was established using 10 μl of plain PBS. BrdU labeling was positive in 35±4% of the HUVECs cultured in VEGF−MSC conditioned media, but was essentially absent in HUVECs cultured in lacZ-MSC conditioned media. This suggested that the VEGF expressed by the transduced MSCs was functional.

### Example 19

### VEGF−MSCs and VEGF−MSCs Conditioned Culture Medium Decreases Apoptosis in Cultured HL-1 Myocytes

The VEGF−MSCs conditioned culture medium significantly decreased HL-1 myocytes apoptosis. Similarly, co-culture of VEGF−MSCs with HL-1 myocytes substantially decreased HL-1 myocytes apoptosis. The anti-apoptotic effects of VEGF−MSCs conditioned media were blocked by the addition of a VEGF antibody to the media. These data indicate that VEGF−MSCs secrete an anti-apoptotic substance which appears to be VEGF.

### Example 20

### Transplantation of VEGF−MSCs Improves Cardiac Function

It was hypothesized that the transplantation of autologous mesenchymal stem cells engineered to overexpress VEGF into hearts with severe concentric LVH would instigate reparative responses in the myocardium to subsequently improve contractile performance, confer resistance to myocardial bioenergetic abnormalities and prevent the transition to heart failure.

Nine of the 19 subjects in the untreated LVH group developed ascites (100-1000 ml), suggesting the presence of biventricular decompensation. Therefore, the untreated LVH group was divided into two LVH and CHF subgroups based on the presence or absence of ascites, respectively. The LV weight to body weight ratio (LVW/BW; g/kg) increased by ˜50% in the untreated LVH group without ascites as well as in the MSCs transplanted groups (none of which developed ascites; p<0.05, Table 2). In contrast, LVW/BW was increased by 118% in CHF hearts (p<0.01 vs. LVH and N, Table 2). Concordantly, the RVW/BW was also significantly increased in LVH groups, which was also most severe in CHF hearts (p<0.05, Table 2). Hence, both MSCs and VEGF−MSCs transplantation attenuated the progression of LVH and prevented the development of the LV decompensation that was present in 49% of untreated pressure overloaded hearts.

TABLE 2

| | | | | | LV Weight/Body | RV Weight/Body |
|---|---|---|---|---|---|---|
| Group | n | Body Weight (kg) | LV Weight (g) | RV Weight (g) | Weight (g/kg) | Weight (g/kg) |
| Normal | 8 | 40.5 ± 2.5 | 91.8 ± 10.3 | 33.3 ± 1.8 | 2.21 ± 0.23 | 0.83 ± 0.05 |
| LVH | 10 | 45.0 ± 2.0 | 157.0 ± 22.1# | 49.8 ± 6.9# | 3.80 ± 0.33# | 1.10 ± 0.12 |
| CHF | 9 | 35.7 ± 6.1 | 155.3 ± 21.6#^{ab} | 61.9 ± 7.7#^{ab} | 4.82 ± 0.66#^{ab} | 1.99 ± 0.41#^{abc} |
| LVH + | 8 | 39.8 ± 3.0 | 139.3 ± 7.3# | 46.5 ± 4.8 | 3.50 ± 0.11# | 1.18 ± 0.09 |
| MSCs | | | | | | |
| LVH + | 7 | 36.8 ± 5.4 | 121.8 ± 17.7 | 41.1 ± 6.0 | 3.34 ± 0.10#* | 1.12 ± 0.05 |
| VEGF- | | | | | | |
| MSCs | | | | | | |

Values are mean ± SEM; n, number of pigs; MSCs, mensenchymal stem cells; VEGF, vascular endothelial growth factor;
*p < 0.05 vs normal;
#p < 0.01 vs. normal;
^{a}p < 0.05 vs LVH + MSCs;
^{b}p < 0.05 vs. LVH + VEGF-MSCs;
^{c}p < 0.05 vs. LVH.

Hemodynamic data are summarized in Table 3. The heart rate and distal mean aortic pressure were not significantly different between the groups (Table 3). The LVH groups had significantly elevated LV systolic pressures during basal conditions as expected (Table 3). Only the CHF group showed a significantly increased LV end-diastolic pressure (p<0.01, Table 3). During the HCW induced by the combined dobutamine and dopamine infusion all groups increased heart rate and LV systolic pressure (Table 3). During HCW, the LV systolic pressure and RPP were remarkably higher in LVH+VEGF−MSCs hearts as compared to other LVH groups and the normal control group (P<0.01, Table 3). Contractile reserve as defined by the percent increase of rate pressure product (RPP) between baseline and HCW [Contractile Reserve (CR %)=100%×(RPP_{hw}−RPP_{baseline})/RPP_{baseline})], was significantly reduced in non-transplanted LVH hearts and the reduction was most severe in the CHF group. However, the fall of CR was partially attenuated in LVH+MSCs hearts, and completely abrogated in LVH+VEGF−MSCs hearts (p<0.05). The LV systolic wall thickening fraction measured by MRI was not significantly different under the basal conditions between the 4 groups although it trended lower in the LVH−CHF group (Table 3). In response to high dose catecholamine stimulation the systolic wall thickening fraction increased in all groups, the response was greater in hearts which received MSCs and was most prominent in hearts that received VEGF−MSCs (p<0.05, Table 3).

TABLE 3

| | | | | | | | | |
|---|---|---|---|---|---|---|---|---|
| | | | | | | RPP 1000x | | LV Systolic |
| | | HR | MAP | LVSP | LVEDP | (mmHg | Contractile | Thickening |
| Group | n | (beats/min) | (mmHg) | (mmHg) | (mmHg) | beats/min) | Reserve (%) | Fraction (%) |

Baseline

| | | | | | | | |
|---|---|---|---|---|---|---|---|
| 8 | 111 ± 9 | 78 ± 7 | 108 ± 4 | 6 ± 1 | 12.21 ± 1.35 | — | 14 ± 2 |

Normal

| | | | | | | | | |
|---|---|---|---|---|---|---|---|---|
| LVH | 10 | 114 ± 6 | 96 ± 7 | 145 ± 15* | 11 ± 3*§^{a} | 16.56 ± 1.82* | — | 15 ± 2 |
| CHF | 9 | 136 ± 10 | 85 ± 9 | 137 ± 12* | 17 ± 3†§#^{a} | 18.65 ± 2.19* | — | 11 ± 3 |
| LVH + | 8 | 121 ± 12 | 91 ± 8 | 139 ± 7* | 6 ± 1 | 7 ± 1* | — | 14 ± 3 |
| MSC | | | | | | | | |
| LVH + | 7 | 133 ± 10 | 93 ± 7 | 132 ± 9* | 7 ± 1 | 18.05 ± 2.12* | — | 16 ± 2 |
| MSC- | | | | | | | | |
| VEGF | | | | | | | | |

DbDP

| | | | | | | | | |
|---|---|---|---|---|---|---|---|---|
| Normal | 8 | 169 ± 13† | 92 ± 6 | 175 ± 7† | 6 ± 1 | 29.58 ± 2.54† | 162.56 ± 37.72 | 21 ± 2† |
| LVH | 10 | 168 ± 11† | 89 ± 6 | 200 ± 24† | 11 ± 2 | 33.24 ± 3.45*† | 101.68 ± 12.89* | 19 ± 2† |
| CHF | 9 | 179 ± 19† | 86 ± 8 | 186 ± 22† | 18 ± 3§#^{a} | 33.28 ± 6.73*† | 82.04 ± 24.41§^{a} | 15 ± 3§^{a} |
| LVH + | 8 | 176 ± 10† | 79 ± 7 | 205 ± 22† | 12 ± 3 | 35.60 ± 2.27*† | 119.26 ± 16.52* | 26 ± 2*† |
| MSC | | | | | | | | |
| LVH + | 7 | 181 ± 14† | 79 ± 10 | 277 ± 31*†#§^{b} | 9 ± 1 | 49.22 ± 4.96*†#§^{b} | 168.15 ± 20.59*#§ | 32 ± 4*††#§ |
| MSC- | | | | | | | | |
| VEGF | | | | | | | | |

Values are mean ± SE; n, number of pigs;
†p < 0.05 vs. baseline;
*p < 0.05 vs. normal;
#p < 0.01 vs. LVH;
§p < 0.05 vs. LVH + MSCs;
^{a}p < 0.05 vs. LVH + VEGF-MSCs;
^{b}p < 0.05 vs. CHF;
HR, heart rate; MAP, Mean aortic pressure; LVSP, LV systolic pressure; LVEDP, LV endo-diastolic pressure; RPP, rate pressure product; CR, Contractile Reserve = 100% × (RPP_{DbDp} − RPP_{baseline})/RPP_{baseline}; DbDp, dobutamine and dopamine (20 μg/kg/min i.v.).

The regional myocardial blood flow data are summarized in Table 3. In both the anterior and posterior walls, basal state blood flows were moderately (but significantly; p<0.05) higher in LVH+VEGF−MSCs group than the other groups (Table 4). At HCW, myocardial blood flow rose substantially in all groups (p<0.05, Table 4). This increase of MBF was significantly higher in LVH+VEGF−MSC group (p<0.05, Table 4).

TABLE 4

| | | LAD Region | | | LCx Region | | |
|---|---|---|---|---|---|---|---|
| | n | EPI | MID | ENDO | EPI | MID | ENDO |
| Baseline | | | | | | | |
| Normal | 8 | 0.66 ± 0.08 | 0.77 ± 0.07 | 0.76 ± 0.06 | 0.68 ± 0.09 | 0.76 ± 0.08 | 0.74 ± 0.09 |
| LVH | 10 | 0.62 ± 0.06 | 0.80 ± 0.10 | 0.75 ± 0.11 | 0.62 ± 0.09 | 0.79 ± 0.10 | 0.72 ± 0.07 |
| CHF | 9 | 0.66 ± 0.09 | 0.72 ± 0.11 | 0.71 ± 0.13 | 0.74 ± 0.11 | 0.74 ± 0.09 | 0.69 ± 0.09 |
| LVH + | 8 | 0.67 ± 0.09 | 0.81 ± 0.08 | 0.74 ± 0.08 | 0.60 ± 0.07 | 0.84 ± 0.10 | 0.82 ± 0.11 |
| MSCs | | | | | | | |
| LVH + | 7 | 0.89 ± 0.08*^{abc} | 1.14 ± 0.09*^{abc} | 1.04 ± 0.06*^{abc} | 0.78 ± 0.07 | 1.12 ± 0.08*^{abc} | 1.13 ± 0.10*^{abc} |
| VEGF- | | | | | | | |
| MSCs | | | | | | | |
| DbDp | | | | | | | |
| Normal | 8 | 1.33 ± 0.13† | 1.32 ± 0.16† | 1.20 ± 0.15† | 1.50 ± 0.16† | 1.54 ± 0.19† | 1.42 ± 0.13† |
| LVH | 10 | 1.24 ± 0.12† | 1.50 ± 0.18† | 1.34 ± 0.21† | 1.39 ± 0.11† | 1.75 ± 0.17† | 1.62 ± 0.12† |
| CHF | 9 | 1.25 ± 0.14† | 1.22 ± 0.18† | 1.14 ± 0.18† | 1.29 ± 0.10† | 1.22 ± 0.13† | 1.18 ± 0.13† |
| LVF + | 8 | 1.32 ± 0.15† | 1.48 ± 0.19† | 1.31 ± 0.20† | 1.46 ± 0.11† | 1.72 ± 0.12† | 1.70 ± 0.14† |
| MSCx | | | | | | | |
| LVH + | 7 | 1.93 ± 0.13†*^{abc} | 1.95 ± 0.22†*^{ab} | 1.82 ± 0.18†*^{abc} | 1.83 ± 0.12†*^{abc} | 2.13 ± 0.14†*^{b} | 1.86 ± 0.12†*^{b} |
| VEGF- | | | | | | | |
| MSCs | | | | | | | |

Values are means ± SEM; n, number of pigs;
*p < 0.05 vs. normal;
^{a}p < 0.5 vs. LVH;
^{b}p < 0.05 vs. CHF;
^{c}p < 0.05 vs. LVH + MSCx;
†p < 0.01 vs. baseline;
DbDp, Dobutamine and Dopamine (20 μg/kg/min i.v.); EPI, Epicardial layer; MID, Midmyocardial layer; ENDO, Endocardial layer

All HEP data from whole LV wall spectra of each group are summarized in Table 5. The basal state PCr/ATP ratios of all LVH groups were significantly lower than those of the normal groups and this reduction was most severe in LVH−CHF hearts. During HCW the PCr/ATP was further significantly reduced in all groups except for the animals that received VEGF−MSCs transplantation. Surprisingly, the latter group maintained baseline PCr/ATP values during HCW (Table 5) despite the fact that they expended ˜40% more energy (as reflected in the RPP data shown in Table 4) than achieved by the group with the next highest RPP. Transmurally differentiated spectra obtained at baseline and during HCW indicated that these reductions were most prominent in the subendocardial layers of all groups. During HCW, myocardial inorganic phosphate levels (expressed as ΔPi/PCr) rose (Table 5). This increase was markedly attenuated in the LVH group receiving VEGF−MSCs and was moderately reduced in the MSCs group (Table 5). These data indicate that MSCs transplantation improves the bioenergetic response to HCW in pressure-overloaded myocardium (Table 5).

TABLE 5

| | Normal | LVH | CHF | LVH + MSCs | LVH + VEGF-MSCs |
|---|---|---|---|---|---|
| | ΔPi/PCr | | | | |
| N | 8 | 10 | 9 | 8 | 7 |
| Baseline | 0 | 0 | 0 | 0 | 0 |
| DbDp | 0.26 ± 0.09# | 0.23 ± 0.08# | 0.50 ± 0.14# | 0.17 ± 0.08# | 0.04 ± 0.05*§ |
| | PCr/ATP | | | | |
| N | 8 | 10 | 9 | 8 | 7 |
| Baseline | 2.04 ± 0.09 | 1.83 ± 0.09† | 1.68 ± 0.08† | 1.88 ± 0.08† | 1.85 ± 0.09† |
| DbDp | 1.82 ± 0.12# | 1.63 ± 0.09†# | 1.60 ± 0.07†# | 1.74 ± 0.07# | 1.81 ± 0.08§ |

Values are mean ± SEM; n, number of pigs; PCr, phosphocreatine;
*p < 0.05 vs. LVH;
†p < 0.05 vs. normal;
§p < 0.05 vs CHF;
#p < 0.05 vs. baseline.

The engrafted cell number was analyzed by X-gal and DAPI double positive nuclei in every 10^{th} serial sections. By week 4, there are no significant differences of cell engraftment rate in MSC alone and VEGF−MSC transplantation hearts (MSC alone, 1.81±0.43%; VEGF−MSCs, 2.06±0.27%; n=4, p>0.05).

Taken together, these experimental findings show that transplantation of VEGF-overexpressing MSCs in severe LVH improved contractile performance and myocardial bioenergetics, reduced cardiac hypertrophy, and prevented the transition to heart failure.

### Example 21

### Transplanted MSCs Developed Into Cardiomyocyte-Like Cells and Promoted Angiogenesis/Neovascularization

Immunohistology assessed the contribution of engrafted VEGF−MSCs in the host myocardium and provided a cellular basis for explaining the functional improvements. H&E and X-gal stainings of cell-treated LVH hearts showed β-galactosidase-expressing cells populating the myocardium, with the majority of cells appearing to have homed to the left ventricular anterior wall and aligned parallel to host cardiomyocytes. Interestingly, the well-defined cross striations can be seen clearly in β-galactosidase-expressing cells, which were also co-stained with alpha sarcomeric myosin heavy chain antibody. Double staining for β-galactosidase and cardiac-specific proteins showed that β-galactosidase was expressed in cardiac troponin T and phospholamban-positive cardiomyocytes. These observations suggest that the transplanted VEGF−MSCs could transdifferentiate into cardiomyocyte-like cells.

Engrafted VEGF−MSCs were examined to determine whether or not they could induce angiogenesis and neovascularization, and transdifferentiate into vascular cells. Immunofluorescence staining for von Willebrand factor (vWF) indicated significant angiogenesis in VEGF−MSCs treated hearts, with more vWF-expressing capillaries in the cell-treated LVH hearts compared to control. The VEGF−MSC treated LVH group had a mean number of vWF⁺ capillaries per high power field of 42±5 compared to 32±2 for the normal and 27±3 for the untreated LVH groups (n=6, P<0.01). Real-time quantitative RT-PCR revealed a significant increase in vWF mRNA expression in the cell-treated LVH hearts compared to normal and untreated LVH hearts (normal heart 1.90±0.19; untreated LVH heart 1.39±0.33; cell-treated heart 4.49±0.76; n=5, P<0.01). There were no significant differences in capillary numbers and vWF mRNA expression between untreated LVH and normal hearts. VEGF mRNA expression in cell-treated hearts was higher compared to both untreated LVH and normal hearts (normal heart 1.20±0.12; untreated LVH heart 1.39±0.33; cell-treated treated heart 3.98±0.38; n=5, P<0.001). Interestingly, double staining clearly showed that β-galactosidase positive nuclei were colocalized with endothelial cell marker caveolin-1. These results indicate that transplantation of VEGF−MSCs into severe LVH induces angiogenesis and neovascularization by stimulating the proliferation of endogenous vascular or vascular progenitor cells. Moreover, transdifferentiation of transplanted MSCs into vascular cells also might be involved in the increases in angiogenesis and neovasculization. Taken together, these findings suggest that the increased neovascularization in response to the cellular therapy improved myocardial perfusion to both engrafted VEGF−MSCs and spared host cardiomyocytes, thereby improving LV contractile performance.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device for occluding blood flow in a vessel, comprising:
a catheter body, the catheter body having a proximal portion and a distal portion, wherein the proximal and distal portions define a longitudinal axis, wherein the catheter body has an inner lumen; and
an occlusion member, wherein the occlusion member comprises a bore through which the catheter body is engaged, wherein the occlusion member comprises a distal end and a proximal end.

2. The device of claim 1, further comprising: an outer sheath, wherein the outer sheath is slidably movable along the longitudinal axis of the catheter body.

3. The device of claim 1, further comprising: a catheter body sheath, wherein the catheter body sheath is slidably movable along the longitudinal axis of the catheter body, wherein the catheter body sheath is located between the catheter body and the outer sheath, wherein the distal end of the catheter body sheath is engaged with the proximal end of the occlusion member.

4. The device of claim 3, wherein the catheter body sheath further comprises a catheter body sheath flange, wherein the flange is engaged with the proximal end of the occlusion member.

5. The device of claim 1, wherein the occlusion member is cylindrically-shaped or conically-shaped.

6. The device of claim 1, wherein the occlusion member is silicone or foam.

7. The device of claim 1, wherein the occlusion member is biodegradable.

8. The device of claim 1, wherein the occlusion member is impregnated with a bioactive substance.

9. The device of claim 8, wherein the bioactive substance is selected from the group consisting of one or more chemotherapeutic agents, one or more growth factors, or a nucleic acid encoding a growth factor.

10. The device of claim 1, wherein the device is disposable.

11. The device of claim 1, wherein the device is sterilizable.

12. The device of claim 1, wherein the inner lumen of the catheter body comprises a composition selected from the group consisting of stem cells, one or more growth factors, one or more chemotherapeutic agents, a nucleic acid encoding a growth factor, and an anti-inflammatory compound.

13. A method of delivering a composition to a biological target in an individual, comprising:
inserting and advancing the distal portion of the catheter body of the device of claim 1 into a vessel in the vasculature of the individual such that the distal portion of the device is positioned at a delivery site in the vessel;
retracting the outer sheath such that the occlusion member substantially occludes blood flow through the vessel;
delivering the composition to the delivery site via the inner lumen of the catheter body; and
removing the device from the vessel.

14. The method of claim 13, further comprising:
discharging the occlusion member from the distal portion of the catheter body, wherein the discharging is prior to the removing step.

15. The method of claim 13, wherein the occlusion member is foam, silicone, or biodegradable.

16. The method of claim 13, wherein the occlusion member is cylindrically-shaped or conically-shaped.

17. The method of claim 13, wherein the vessel is a vein.

18. The method of claim 13, wherein the composition is selected from the group consisting of stem cells, one or more growth factors, one or more chemotherapeutic agents, a nucleic acid encoding a growth factor, and an anti-inflammatory compound.

19. The method of claim 18, wherein the stem cells comprise a heterologous nucleic acid encoding VEGF.

20. The method of claim 13, wherein the biological target is selected from the group consisting of heart, liver, pancreas, kidney, brain, uterus, ovaries, prostate, testicles, intestines, eyes, vocal chord, and solid cancer tumor.

21. The method of claim 14, wherein the bore in the occlusion member closes following discharging of the occlusion member.

22. The method of claim 13, wherein the delivery site in the vessel is distal to the occlusion member.
